(19) United States
(12) Patent Application Publication
(10) Pub. No.: US 2025/0345275 A1
(43) Pub. Date: Nov. 13, 2025

(54) Micromolar Halogenated Fluorescein Assists in Full Skin-Thickness Wound Healing

(71) Applicants: Provectus Pharmatech, Inc., Knoxville, TN (US); Board of Regents, The University of Texas System, Austin, TX (US)

(72) Inventors: Amina EL AYADI, Galveston, TX (US); Jayson Walter JAY, Houston, TX (US); Eric A. WACHTER, Oak Ridge, TN (US); Dominic RODRIGUES, Knoxville, TN (US); Edward V. PERSHING, Knoxville, TN (US)

(73) Assignees: Provectus Pharmatech, Inc., Knoxville, TN (US); Board of Regents, The University of Texas System, Austin, TX (US)

(21) Appl. No.: 19/198,639

(22) Filed: May 5, 2025

Related U.S. Application Data
(60) Provisional application No. 63/645,555, filed on May 10, 2024.

(51) Int. Cl. A61K 9/10; A61K 9/00; A61K 31/353; A61K 47/34; A61P 17/02

(52) U.S. Cl. CPC A61K 9/10; A61K 9/0014; A61K 31/353; A61K 47/34; A61P 17/02

(57) ABSTRACT

The present invention contemplates a method of treating a mammalian skin wound that extends at least into the epidermal layer of the skin of a subject mammal that comprises treating the wound in the substantial absence of actinic light by topical application of an aqueous pharmaceutical composition containing a wound closure-assisting amount of rose bengal, a lactone, salt, ester or amide hereof dissolved or dispersed therein as well as gel-inducing amount of a thickening agent (gellant) that causes the aqueous pharmaceutical composition to gel at a temperature of about 33° to about 40° C. The treated wound is thereafter covered with a dressing that is opaque to actinic light at least in the area over the wound. This treatment method is repeated a plurality of times over the following up to about fifteen days or until the wound is closed, whichever time of time is shorter.

### CROSS-REFERENCE TO RELATED APPLICATION

This application claims priority to U.S. application Ser. No. 63/645,555, filed on May 10, 2024, whose disclosures are incorporated herein by reference.

### BACKGROUND ART

The skin is the largest organ in the body and covers the body's entire external surface. It is made up of three primary layers, the epidermis, dermis, and the hypodermis, all three of which vary significantly in their anatomy and function. The skin's structure is made up of an intricate network that serves as the body's initial barrier against pathogens, UV light, and chemicals, and mechanical injury. It also regulates temperature and the amount of water released into the environment.

The epidermis has five layers of cells, of which, the uppermost (outermost) layer is called the stratum corneum and itself contains several cell layers of which the uppermost is made of keratin and horny scales made of dead keratinocytes. The deepest (innermost) layer of the epidermis is the stratum basale is separated from the dermis by the basement membrane (basal lamina). The cells found in this layer are cuboidal to columnar mitotically active stem cells that are constantly producing keratinocytes. This layer also contains melanocytes.

The dermis has two layers that merge together without a clear demarcation. The papillary layer is the upper layer: thinner, and composed of loose connective tissue and contacts epidermis. The reticular layer is the deeper layer: thicker, less cellular, and consists of dense connective tissue/bundles of collagen fibers. The dermis houses the sweat glands, hair, hair follicles, muscles, sensory neurons, and blood vessels.

The hypodermis is beneath the dermis and is also called subcutaneous fascia. It is the deepest layer of skin and contains adipose lobules along with some skin appendages like hair follicles, sensory neurons, and blood vessels.

A wound is defined as an opening in the skin as the result of trauma, pressure or surgery. Broadly-speaking there are four types of wounds. Abrasions are made when the skin is rubbed or scraped off. Rope burns, rug burns, and skinned knees or elbows are common examples of abrasions. Incisions, commonly called cuts, are wounds made by sharp cutting instruments such as knives, razors, scalpels, and broken glass. Incisions tend to bleed freely because the blood vessels are cut cleanly and without ragged edges. Lacerations are wounds that are torn, rather than cut. They have ragged, irregular edges and masses of torn tissue underneath. These wounds are usually made by blunt, rather than sharp, objects. A wound made by a dull knife, for instance, is more likely to be a laceration than an incision. Punctures are caused by objects that penetrate tissues while leaving a small surface opening. Wounds made by nails, needles, and bullets are usually punctures. [Meyers, et al., Wound Care: Getting to the Depth of the Tissue, National Center of Continuing Education, Inc., 1-16 (2013).]

To assist the wounded subject, the care-giver must go through several assessments of the subject and of the wound itself. One of those assessments is the staging of the wound.

Stage 1 (partial thickness) is defined as a reddened area or non-blanchable erythema over a bony prominence. This area can be painful, firm, soft, warmer or cooler compared to surrounding tissue. Stage 2 (partial thickness) is the removal of the first two layers of tissues from the epidermis, and includes the dermis. This includes a fluid or sanguineous filled blister. Stage 3 (full thickness) is defined as progressing to the subcutaneous fat layer. There is no tendon or muscle. Stage 4 (full thickness) is defined as damage to issue that has proceeded to expose the bone. Slough, eschar, tunnels, and undermining may be present. [Meyers et al., supra, at 7.]

As is seen by the above staging classifications, there is some overlap between stages as to which of the layers of skin has been damaged. For that reason, rather than using the above or similar staging criteria, the depth of the wound is used to distinguish the wound types. Thus, is the epidermis the only tissue observable tissue damaged? Are both the epidermis and dermis ae damaged, but not the hypodermis? Thirdly, all three skin layers damaged? Such damage is referred to herein as full skin-thickness when any portion of the wound includes the hypodermis.

Wound healing, particularly in full skin-thickness injuries, presents a significant clinical challenge. Traditional approaches often yield limited success, highlighting the need for innovative treatments.

The present inventors and co-workers recently reported that rose bengal photodynamic therapy accelerates wound closure and improves wound healing [2022 Annual Meeting of the Wound Healing Society SAWC-Spring/WHS joint Meeting, Apr. 6-10, 2022, Phoenix, AZ, Published Mar. 3, 2022]. That study utilized rose bengal purchased from a chemical supply house that had some unknown impurities and was not of medical grade. RB photoactivation was achieved by irradiating cells with 550 nm monochromatic green light (MGL) using a 50 W LED flood light (Loftek, Newark, CA, USA).

The present study examines the therapeutic potential of a medical grade, but not yet approved for human use grade of rose bengal (RB), referred to as PV-10®, in enhancing wound healing processes. PV-100 was kindly provided by Provectus Biopharmaceuticals, Inc., of Knoxville, TN. Because photoactivation may not be available in every clinical setting, the efficacy and safety of a single dose versus multi-dose RB application under monochromatic green light (MGL) and ambient light, respectively, were compared.

### BRIEF SUMARY OF THE INVENTION

The present invention concerns rose bengal-assisted skin wound closure (healing) that is topically applied in the substantial absence of actinic light. More specifically, the invention contemplates a method of treating a mammalian skin wound that extends at least into the epidermal layer of the skin of a subject mammal that comprises treating the wound in the substantial absence of actinic light by topical application of an aqueous pharmaceutical composition containing a wound closure-assisting amount of rose bengal dissolved or dispersed therein as well as a gel-inducing amount of a thickening agent (gellant) that causes the aqueous pharmaceutical composition to gel at a temperature of about 330 to about 40° C. The treated wound is thereafter covered with a dressing that is opaque to actinic light at least in the area over the wound.

A preferred aqueous pharmaceutical composition is a hydrogel (gel) at a temperature of about 33° to about 40° C., that is, the body temperature of the wounded subject receiving the treatment. More preferably, a contemplated aqueous pharmaceutical composition is a liquid (sol) at a temperature of about 15° to about 26° C., and a gel at a temperature of about 33° to about 40° C.

Typically, a preferred aqueous pharmaceutical composition is maintained at a temperature below the gelation temperature, so that the composition is applied as a liquid. Shortly after application, the composition preferably gels in place due to the wounded subject's body temperature being above the gelation temperature of the composition.

The gellant of a contemplated aqueous pharmaceutical composition constitutes a total about 15 to about 25, and preferably about 17 to about 22, weight percent of the composition. That gellant can be a single hydrogel-forming (or gel-forming) polymer (gelling agent) as the thickening agent, or a mixture of a principal gellant along with one or more other secondary gellants.

A primary gellant constitutes about 80 to about 100 weight percent of the total gellant, whereas the one or more secondary gellants, combined, constitute zero to about 20 percent of the total weight of gellant. Thus, for example, an aqueous pharmaceutical composition containing 20 weight percent total gellant, that total weight percentage can include 16 weight percent primary gellant and 4 weight percent of one or more secondary gellants. One or more secondary gellants, when present constitute about 0.1 to about 5 weight percent of the total weight of a contemplated aqueous pharmaceutical composition.

After topical application of the above aqueous pharmaceutical composition, the treated wound is covered with a dressing that is opaque to actinic light in the area over the wound. This treatment method is repeated a plurality of times over the following up to about fifteen days or until the wound is closed, whichever period of time is shorter.

### BRIEF DESCRIPTION OF THE DRAWINGS

In the drawings forming a portion of this disclosure and in which one or more asterisks (*) is shown, one asterisk (*) indicates a significant difference at p<0.05, whereas two asterisks (**) indicate a significant difference at p<0.01.

FIG. 1 in two panels, as FIGS. 1A and 1B, are graphs in which the areas bounded by doted lines are reference ranges published by Charles Rivers Laboratories for male BALB/c mice, FIG. 1A illustrates the approximately 5% loss of body weight for the test animals that received vehicle plus irradiation (MGL), rose bengal sodium (RBS) present at 0.1% without irradiation and RBS present at 0.1% with irradiation measured at 3 and 7 days post-injury, whereas FIG. 1B illustrates the constant weights of the test animals that received vehicle only or vehicle containing 0.01% RB days 3 and 7 post-injury, and slight weight gains at day 14 post-injury;

FIG. 2 in two panels, as FIGS. 2A and 2B, are graphs in which the areas bounded by doted lines are reference ranges published by Charles Rivers Laboratories for male BALB/c mice, FIG. 2A illustrates the TEWL for animals treated only with vehicle and irradiation (MGL), rose bengal sodium (RB or RBS) present in vehicle at 0.1% without irradiation and RB present in vehicle at 0.1% with irradiation measured at 3 and 7 days post-injury, whereas FIG. 2B shows similar data for the vehicle alone and vehicle plus 0.01% RBS with no irradiation measured at days 3, 7, and 14 post-injury with the difference between the TEWL amounts at day 14 showed the test animals to be back at their usual range and a statistically significant difference between the TEWL value for the vehicle alone and the vehicle plus 0.01% RBS in which p<0.0001.

FIG. 3, also in two panels as FIGS. 3A and 3B, are graphs in which the areas bounded by doted lines are reference ranges published by Charles Rivers Laboratories for male BALB/c mice, FIG. 3A illustrates the erythema index for animals treated only with vehicle and irradiation (MGL), rose bengal sodium (RBS) present in vehicle at 0.1% without irradiation and RBS present in vehicle at 0.1% with irradiation measured at 3 and 7 days post-injury, whereas FIG. 3B shows similar data for the vehicle alone and vehicle plus 0.01% RBS with no irradiation measured at days 3, 7, and 14 post-injury with the difference between the erythema index amounts at day 7 showed the test animals to exhibit a statistically significant difference between the erythema index for the vehicle alone and the vehicle plus 0.01% RBS in which p<0.0001, with the erythema index values at day 14 post-injury for both the animals treated with the vehicle alone and those treated with the vehicle plus 0.01% RBS being not statistically different and very similar to the usual erythema index values;

FIG. 4 is a graph showing the percentage of wound closure at days 3, 7, and 14 days post-injury, with the degree of closure at day three being statistically significantly less for the animals treated with vehicle plus 0.01% RBS and no irradiation as compared to vehicle alone without irradiation;

FIG. 5, also in two panels as FIGS. 5A and 5B, are graphs in which FIG. 5A illustrates the wound collagen fiber densities for animals treated only with vehicle and irradiation (MGL), rose bengal sodium (RBS) present in vehicle at 0.1% without irradiation and RBS present in vehicle at 0.1% with irradiation measured at 3 and 7 days post-injury, whereas FIG. 5B shows similar data for the vehicle alone and vehicle plus 0.01% RBS with no irradiation measured at days 3, 7, and 14 post-injury with the difference between the erythema index amounts at day 7 showed the test animals to exhibit a statistically significant difference between the erythema index for the vehicle alone and the vehicle plus 0.01% RBS in which p<0.0001, with the erythema index values at day 14 post-injury for both the animals treated with the vehicle alone and those treated with the vehicle plus 0.01% RBS being not statistically different and very similar to the usual erythema index values

FIG. 6 is a graph showing the number of blood vessels noted in a photomicrograph of the wound area in which the blood vessels were stained with labeled antibodies to smooth muscle actin, in which the vehicle alone, the vehicle plus 0.01% RBS was studied at days 3, 7, and 14 post-injury are shown as are results for uninjured tissue plus 0.01% RBS, in which the number of blood vessels on day 7 was statistically greater [p<0.05] than the number at day 3 post-injury, along with additional statistically significant differences shown by the lines with stars between the several conditions.

FIG. 7 is a graph of partial Trichrome staining analysis of the results on day 7 of the study, in units of pixels showing the stain and in which: “Veh”=“vehicle”; “MGL”=“monochromatic green light” (λ=532 nm); “RB”=“rose bengal” present at a stated amount; “MD”=“multiple dosing”; and “Sham+RB MD”=Sham+multiple dosing rose bengal.

### DEFINITIONS

The articles “a” and “an” are used herein to refer to one or to more than one (i.e., to at least one) of the grammatical object of the article. By way of example, “an element” means one element or more than one element. Each of the patents, patent applications, and articles cited herein is incorporated by reference.

Gel and Hydrogel—The Oxford Dictionary of Biochemistry and Molecular Biology, Smith et al eds., Oxford University Press, New York, 296 and 312, (1997) respectively define a gel as “1 a colloidal system, with the semblance of a solid, in which a solid is dispersed in a liquid. A gel has a finite usually rather small, yield stress;” and “[a] hydrogel (def. 1) in which water is the liquid component.” A “sol” is a liquid colloidal system. Similar definitions are found in Stedman's Medical Dictionary, Houghton Mifflin Company, Boston, 329 and 384 (2002) and Merriam-Webster's Medical Desk Dictionary, Merriam-Webster, Incorporated, Springfield, 305 and 361 (2002).

A hydrogel useful in the present invention is formed from a polymeric material characterized by a three-dimensional network that can retain a large amount of water or biological fluid under physiological conditions. These materials can be used as delivery systems due to the unique properties of sol-gel conversion that is modulated by a specific biological stimulus [Giuliano et al., Pharmaceutics 10, 159 page 1 of 26 (2018)].

A particular sol-gel conversion property of a preferred hydrogel-forming polymer is thermoreversibility. A preferred gellant changes from sol to gel and back to sol again dependent upon temperature [Goyal et al., Res J Pharm Tech 3(3): 700-704 (July-September 2010)]. For example, aspics and other gelatin-based hydrogels are formed as sols at a relatively high temperature and form gels when refrigerated temperatures. A preferred gellant is a sol (liquid) at low temperature and gels at an elevated temperature. Reheating a gelled aspic reforms the sol (liquid) form and cooling a preferred hydrogel reforms a liquid.

As used herein, the phrase “actinic light” denotes light that can cause a photochemical reaction of one or more of the ingredients of topical ophthalmic composition. In accordance with this definition, the actinic light is of a wavelength that is absorbed by a recipient molecule of the composition and there is a sufficient flux of photons of the absorbed wavelength to cause a detectable chemical reaction induced in or by the absorbing recipient halogenated fluorescein molecule. Illustrative chemical reactions include decomposition, and photosensitization.

The term “substantial absence of actinic light” is used herein to mean that, during and after preparation of the aqueous pharmaceutical composition, as well as after application of that composition to the wound (treatment), the treated wound is not subjected to sunlight or other intense source of light. It is preferred that the treated wound is subjected to ambient light for a time period of about 2 to about 5 minutes or fewer. Thus, after application, the wound preferably is covered, a light-blocking patch is preferably placed over the treated wound. Room light provided by fluorescent ceiling light fixtures typically does not provide a sufficient photon flux of actinic light for rose bengal (RB) to undergo a chemical reaction. This is thought to be due in part to the low flux of photons as well as the relatively low concentration of RB present in the gelled aqueous pharmaceutical composition, the gel structure itself that can limit molecular motion within the gel, and the relative lack of reactivity of the major constituents of the gel; i.e., water, and the gellant(s).

### DETAILED DESCRIPTION OF PREFERRED EMBODIMENTS

The present invention contemplates a method of treating a skin wound that extends at least into the epidermal layer of the skin of a subject and a composition for the treatment. A contemplated method includes the steps of a) treating the wound in the substantial absence of actinic light by topical application to the wound of an aqueous pharmaceutical composition. That aqueous pharmaceutical composition contains dissolved or dispersed therein i) a wound closure-assisting amount of rose bengal (RB), a pharmaceutically acceptable salt of RB, rose bengal lactone, a RB amide whose nitrogen atom is unsubstituted, substituted with one or two C₁-C₄ alkyl groups that are the same or different or together with the amido nitrogen form a 5- or 6-membered ring, a C₁-C₄ alkyl ester thereof, an aromatic RB derivative, wherein the aromatic derivative is an ester or amide formed from an alcohol or monosubstituted amine having a 5- or 6-membered aromatic ring, or a 5,6- or 6,6-fused aromatic ring system that contains 0, 1 or 2 hetero ring atoms that are independently nitrogen, oxygen or sulfur. A gel-inducing amount of a thickening agent (gellant) that causes the aqueous pharmaceutical composition to gel at a temperature of about 33° to about 40° C. ii) is also present dissolved or dispersed in the aqueous pharmaceutical composition. The thus treated wound is ii) covered with a dressing that is preferably opaque to actinic light at least in the area over the wound. This treatment method is repeated a plurality of times over the following up to about fifteen days or until the wound is closed, whichever time period is shorter.

### Rose Bengal Compounds

Turning to the components of a contemplated aqueous pharmaceutical composition, a contemplated RB compound has the structural formula (Formula I) below, where X is O (oxygen) or N (nitrogen), and “n” is zero or one. When X is oxygen, n is zero

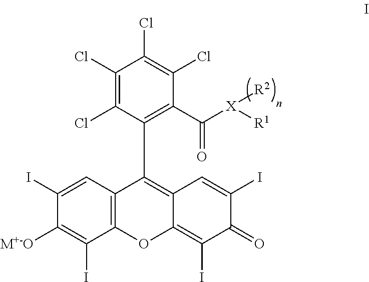

and absent so that the RB compound is a) rose bengal where -X-R¹ is —O—H, b) is a pharmaceutically acceptable salt of RB where X-R¹ is —O⁻ M⁺ and where M⁺ is a pharmaceutically acceptable cation, c) a C₁-C₄ alkyl ester, or is d) an aromatic ester as defined below. The reader is directed to Berge, J. Pharm. Sci. 1977 68(1): 1-19 for lists of commonly used pharmaceutically acceptable acids and bases that form pharmaceutically acceptable salts with pharmaceutical compounds, such as RB. Illustrative cations include alkali metals such as sodium, potassium, as well as ammonium and alkaline earth salts such as magnesium and calcium.

Alternatively, when X is a nitrogen atom, n is 1 and R² is present along with R¹. As such, R¹ and R² can be the same or different and —C(O)-NR¹R² is an amide whose nitrogen atom is a) unsubstituted [-X-(R¹R²) and both R¹ and R² are hydrogen (H)], is b) substituted with one or two C₁-C₄ alkyl groups, or together with the amido nitrogen atom the R¹ and R² alkyl moieties form a 5- or 6-membered ring, or is c) an aromatic amide whose nitrogen atom is preferably monosubstituted in that R¹ is hydrogen and R² is an aromatic substituent discussed below.

For ease of description, an aromatic ester or aromatic amide are collectively referred to herein as an aromatic derivative. As such, those derivatives are formed from an alcohol or amine, preferably monosubstituted, having a single 5- or 6-membered aromatic ring, or a 5,6- or 6, 6-fused aromatic ring system that contains 0, 1, or 2 hetero ring atoms that are independently nitrogen, oxygen or sulfur.

Illustrative examples of such aromatic alcohol ester portions are shown and named below, where O is an oxygen atom and line-O indicates the ring-oxygen can be from any available carbon of the ring and the O-line crossed by a wavy line indicates that the depicted alkoxy or amino group is a portion of another molecule, the esterified RB molecule.

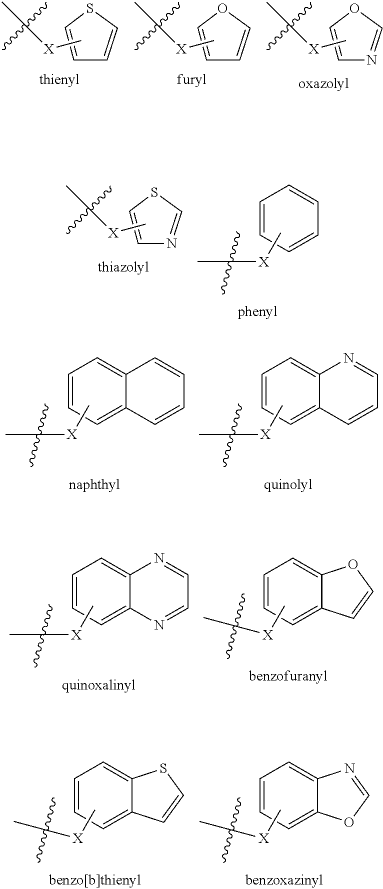

where

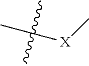

is

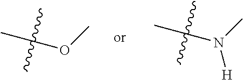

providing an ester or a monosubstituted amine, respectively.

Rose bengal (RB) is a preferred RB compound and its disodium salt, rose bengal disodium (RBD or RBS), is a most preferred RB compound. These compounds are used illustratively herein for the group of RB compounds. The chemical name for rose bengal (RB) is 4,5, 6, 7-tetrachloro-2′, 4′, 5′, 7′-tetraiodo-fluorescein. The structural formula for the preferred rose bengal disodium salt (RBS or RBDS) is shown below.

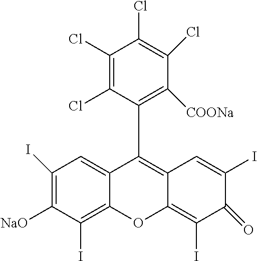

### Gellants

Aqueous hydrogels are well known in the pharmaceutical arts, particularly for use in controlled release compositions. For example, Altomare et al., J Mater Sci Mater Med, 27:95-108 (2016) reported on the preparation of methylcellulose hydrogels whose gelation temperatures could be adjusted using various salts. U.S. Pat. No. 4,615,697 to Robinson teaches the use of a cross-linked polyacrylic acid polymer that now has the name polycarbophil NF, and was used as a bioadhesive to adhere other materials such as resin beads and bovine serum albumin microcapsules to biological surfaces such as the stomach wall, as well as a controlled-release agent for medicaments. Carbomer 934 NF, is another cross-linked polyacrylic acid polymer is similar to polycarbophil, but is water-soluble whereas polycarbophil is water-swellable, but not water soluble. These and other hydrogel-forming materials can be used here, but are preferably used in relatively minor, secondary, amounts along with the particularly preferred primary gellant discussed below.

A particularly preferred primary and/or sole gelling agent is an A-B-A triblock copolymer in which the A block-forming monomers are ethylene oxide and the B block-forming monomers are propylene oxide monomers. On polymerization, these ABA-type triblock copolymers that are composed of polyoxyethylene (A) and polyoxypropylene (B) units, thus forming a class of water-soluble non-ionic triblock copolymer containing a hydrophobic core of polyoxypropylene (POP) between two hydrophilic units of polyoxyethylene (POE).

A particularly preferred principal gelling agent A-B-A triblock copolymer is available under the trade name Pluronic® F-127 and under the National Formulary name poloxamer 407 NF. For this compound (Pluronic® F-127), the A blocks contain about 200 polymerized ethylene oxide units, and the middle B group has an average of about 67 polymerized propylene oxide units. The average molecular weight is said to be about 12,500 Da [Pluronic® F-127, G-Biosciences, St. Louis, MO, downloaded Mar. 13, 2024]. Bodratti et al., J Funct Miomat, 9, 11 (2018) recite a molecular weight of about 12,600 Da, with about 65 polymerized propylene oxide units and about 200 polymerized ethylene oxide units.

A nomenclature adopted to provide useful information about the physico-chemical properties of the various derivatives, according to which each copolymer is characterized by three numbers representing the molecular weight of the hydrophobic portion, and the percentage of the hydrophilic chains. BASF utilizes a specific notation for Pluronic® products. The physical state is specified by a consonant (P: Paste, F: Flake, L: Liquid) followed by two or three digits. For a polymer named as a Pluronic®, such as F68, the first one or two digits, multiplied by 300, indicates the approximate molecular weight of the hydrophobic block [polyoxypropylene (POP) section] of 1800, whereas the percentage amount of polyoxyethylene (POE) is obtained multiplying the last digit by 10, here about 80% [Bodratti et al., J Funct Miomat, 9, 11 (2018)]. For poloxamer-named materials, such as poloxamer 188, which can be another designation for Pluronic® F68, the first one or two numbers from the left multiplied by 100 provides a POP average molecular weight of 1800 Da, and multiplication of the last number by 10 provides the percentage of POE content, in this example approximately 80% POE [Giuliano et al., Pharmaceutics 10:159 26 pages (2018)]. Thus, both calculations arrive at the same values.

Poloxamer molecules form an array of thermodynamically-stable self-assembled structures in solution, driven by differences in the solubility of their constituent PEO and PPO blocks. Individual non-associated block copolymer chains are often termed unimers, to distinguish them from chains, which are organized into supramolecular structures. [Bodratti et al., J. Funct. Biomater. 9:11, 24 pages (2018).]

Because solubility drives self-assembly, the solvent type and temperature are important in determining the system properties. The impacts on self-assembly of block copolymer concentration, molecular weight and PPO/PEO ratio, along with solvent quality, have been explored in many binary (water+poloxamer) and ternary (water+“oil”+poloxamer) systems. Observed self-assembled structures include micelles, reverse (water-in-oil) micelles and other structures.

A particular sol-gel conversion property of a preferred hydrogel-forming polymer is thermoreversibility. A preferred gellant changes from sol to gel and back to sol again dependent upon temperature [Goyal et al., Res J Pharm Tech 3(3):700-704 (July-September 2010)]. For example, aspics and other gelatin-based hydrogels are formed as sols at a relatively high temperature and form gels at cooler temperatures. A preferred gellant is a sol (liquid) at low temperature and gels at an elevated temperature. Reheating a gelled aspic reforms the sol (liquid) form and cooling a preferred hydrogel reforms a liquid.

The one or more secondary gellants can provide one or more of three advantages to the gel formed by the primary gellant. A first advantage is mucoadhesion of the resulting gel to the wound and surrounding skin. The second is to add physical strength to the gel formed from the primary gellant. The third advantage is to adjust the temperature at which the aqueous pharmaceutical composition gels.

Illustrative secondary gellants include chitosan, sodium alginate, gellan gum, k-carrageenan, sodium carboxymethylcellulose, methyl cellulose, hydroxypropyl methylcellulose, hydroxyethyl cellulose, hydroxypropyl cellulose, polyethylene glycol (PEG 400-PEG 4000), polycarbophil, carbomer, and mixtures thereof. The above polymers are seen to be mostly linear and to contain polar side groups dependent from the main polymer backbone such as hydroxyl groups, protonated amines, and carboxylate groups. These secondary gellants are well known and described in the chemical, polymer and patent literature and are commercially available. Giuliano et al., Pharmaceutics 10, 159 pages 8-10 of 26 (2018), discuss several poloxamer compositions containing added secondary gellants.

Further discussions of the use of one or more secondary gellants with poloxamers can be found in: Bilensoy et al., AAPS PharmSciTech 7(2): Article 38 (2006); Rarokar et al., J Food Pharm Sci 5:29-39 (2017); Tirnaksiz et al., Pharmazie 60:518-523 (2005); da Silva et al., Mat Sci Eng C 119:1116432; Altomare et al., J Mat Sci Mat Med 27:95-108 (2016); Giuliano et al., Pharmaceutics, 10:159,26 pages (2018); and Lupu et al., Polymers 15:355 18 pages (2023).

The gellant is typically present in a total amount of about 15 to about 25 weight percent of the aqueous pharmaceutical composition. That weight can be totally comprised of the primary gellant, such as poloxamer 407. More preferably, the primary gellant is present at about 15 to about 20 weight percent.

One or more secondary gellants as noted above can also be present in combination with the primary gellant. The amount of total secondary gellant present can be from about 0.1 weight percent to about 5 weight percent.

Salts such as sodium chloride that are often present in medicaments to provide bodily tonicity are preferably absent or minimized as they tend to disrupt the sol/gel temperature. Illustratively, the addition of 1% of sodium chloride, sodium monohydrogen phosphate or sodium dihydrogen phosphate caused a greater than 60-fold increase in gel strength and over a tenfold increase in bioadhesive force with 2-4° C. decrease of gelation temperature, compared with P 407/P 188 (15/15%) alone [Choi et al., Int J Pharm 1:13-19 (Nov. 10, 1999)]. Non-ionic tonicity builders such as dextrose, mannitol and glycerin can also be used.

Lower alkyl C₂-C₃ alcohols such as ethanol, propylene glycol and glycerin can also be present in a contemplated aqueous pharmaceutical composition. For example, glycerin slightly decreased the gelation temperature and slightly increased the gel strength and bioadhesive force of an aqueous composition of P407/P188 (15/15%). On the other hand, ethanol and propylene glycol increased the gelation temperature and slightly decreased the gel strength and the bioadhesive force. [Choi et al., Int J Pharm 1:13-19 (Nov. 10, 1999).]

A mammalian subject having wound in need of treatment (a mammalian subject) and to which a pharmaceutical composition containing an FX compound or its pharmaceutically acceptable salt or RB disodium can be administered can be a primate such as a human, an ape such as a chimpanzee or gorilla, a monkey such as a cynomolgus monkey or a macaque, a laboratory animal such as a rat, mouse or rabbit, a companion animal such as a dog, cat, horse, or a food animal such as a cow or steer, sheep, lamb, pig, goat, llama or the like.

After topical application of the above aqueous pharmaceutical composition, the treated wound is covered with a dressing that is opaque to actinic light in the area over the wound. Such wound dressings are well known in the art and are used herein as they are normally used in the art to protect the wound from infection, further injury, loss of the administered aqueous pharmaceutical composition, contamination and the like as are known in the art. An illustrative useful dressing is sold under the trademark name ALLEVYN LIFE™ by Smith & Nephew, Inc. of Warsaw, IN. Another useful dressing is sold under the trademark name NEXTCARE™ TEGADERM™ by 3M Company of St. Paul, MN.

### Results

### Weight Loss

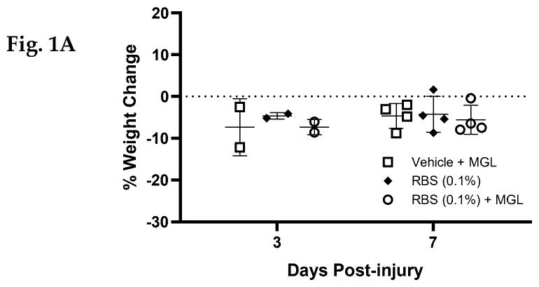

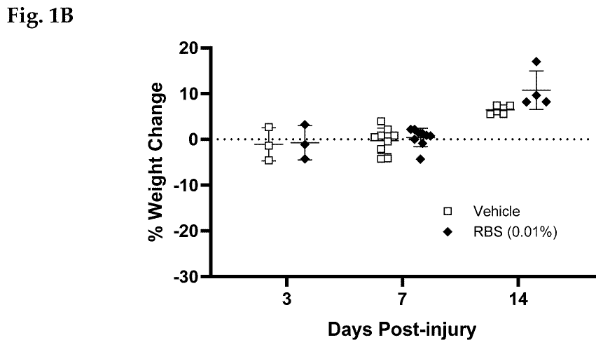

The results of the weight loss study are shown in FIGS. 1A (0.1% RB) and 1B (0.01% RB). As is seen from FIG. 1A, both groups treated with 0.1% RB with and without irradiation, as well as the irradiated control group lost weight over the first seven days post-injury. On the contrary, the control and the 0.01% RB-treated group maintained their weights through the seventh-day post injury and gained weight between the seventh-and fourteenth-day post injury. The basis for this weight discrepancy between the treatment groups is presently unknown.

### CBC

The results from the CBC study showed substantially no differences except in body weight as discussed below. The abbreviations for the individual parameters studied are shown in the table below:

CBC Study

| ASSAY | ABBR* | ASSAY | ABBR* |
|---|---|---|---|
| White blood cells | WBC | Alanine | ALT |
| | | aminotransferase | |
| Lymphocytes | LYMPH | Total bilirubin | tBIL |
| Monocytes | MONO | Total protein | TPRT |
| Neutrophils | NEUT | Globulin | GLOB |
| Red blood cells | RBC | Amylase | AMY |
| Hemoglobin | HGB | Glucose | GLU |
| Hematocrit | HCT | Calcium | CA |
| Mean corpuscle hemoglobin | MCH | Phosphorous | PHOS |
| Mead corpuscle volume | MCV | Alkaline | ALP |
| | | phosphatase | |
| Mean corpuscle hemoglobin | MCHC | Alanine | ALT |
| concentration | | aminotransferase | |
| Platelets | PLT | Sodium | NA |
| Mean platelet volume | MPV | Potassium | K |
| Albumin | ALB | Creatinine | CRE |
| Alkaline phosphatase | ALP | Blood urea nitrogen | BUN |

ABBR* = Abbreviation

For the groups that received 0.1% RB every other day with or without photoactivation:
All animals survived through time course (n=19); no adverse events noted;
About a 5% mean body mass loss out to 7 days in all treatment groups;
Leukocyte count normal out to 7 days post-injury;
Erythrocytes and platelets size, morphology, count normal out to 7 days; and
Elevated mean amylase in all treated groups; other chemistries within normal ranges.

For the group that received 0.01% RB every other day without photoactivation:
All animals survived through time course (n=33), no adverse events noted;
Mean body mass maintained throughout treatment in both groups, with a slight increase with MD RB at 14 days;
Leukocyte count normal out to 14 days post-injury/treatment; Erythrocytes and platelets size, morphology, count normal out to 14 days; and
Elevated mean amylase in all groups, other chemistries within normal ranges.

### Transepidermal Water Loss and Wound Erythema

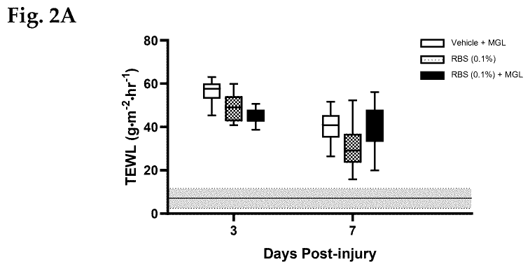

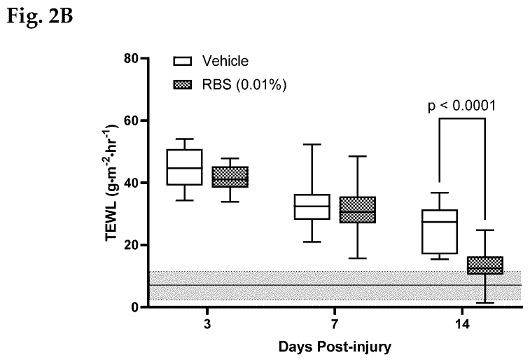

At each time point prior to euthanasia, transepidermal water loss (TEWL) and wound erythema indices were measured using the validated DermaLab® Combo System (Cortex Technologies, Hadsund, Denmark), whereas transepidermal water loss (TWEL) was significantly reduced by 14 days post-injury (p<0.0001) suggesting improvement in the skin barrier function. This is seen particularly in the 0.01% RB treatment as shown in FIG. 2B where the TWEL was about at the normal level before injury at that 14-day post-injury measurement.

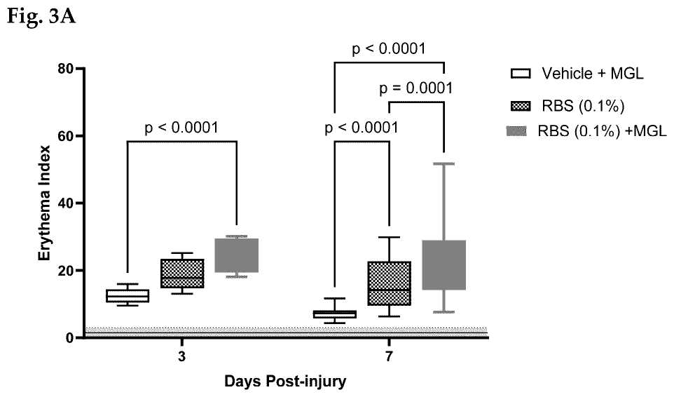

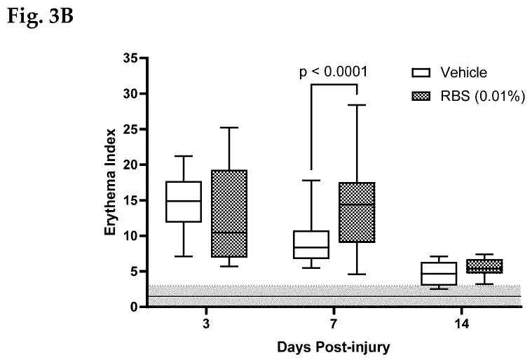

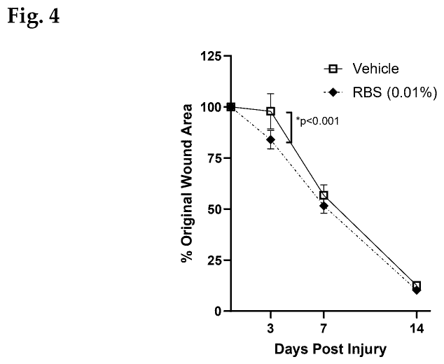

Erythema in the animals treated with 0.1% RB was about the same at days 3 and 7 post injury as is seen in FIG. 3A. To the contrary, erythema was significantly increased on day 7 (p<0.0001) as seen in FIG. 3B.

Animals were placed under general anesthesia and a 10-mm diameter TEWL probe was placed directly over each wound ensuring uniform coverage and forming a seal immediately around the wound. Following environmental normalization to ambient temperature and relative humidity, TWEL was calculated at g m⁻¹ hr⁻¹. A minimum of three TWEL measurements were obtained for each full-thickness wound and uninjured skin area. A color sensitive 7-mm diameter probe, insensitive to ambient light, was then placed over each wound to measure erythema; four measures were obtained for each wound. All wound erythema calculations were normalized to uninjured skin and reported as an index value over time.

Both single and multidose applications of RB to full-thickness wounds did not illicit toxicity. No loss in body or organ weight was observed with either treatment regimen. Secondary outcomes analysis shows that treatment with 0.01% RB accelerates wound closure compared to vehicle alone with a significant difference on day 3 after injury (P<0.001) as is seen in FIG. l .

These data show that topical single-dose photoactivated PV-10, and covered multidose PV-10 under ambient light conditions are both safe for wound healing applications. Both treatment regimens accelerate wound closure and improve skin function suggesting their suitability for clinical application.

### Materials and Methods

### Animal Model of full-Thickness Cutaneous Wounds

Animals: The Institutional Animal Care and Use Committee of the University of Texas Medical Branch in Galveston (UTMB) approved all protocols and studies were conducted in accordance with animal welfare regulations. Healthy 7-week-old male BALB/c mice (N=58) (strain 028, Charles River Laboratories, Houston, Texas, USA) 18-22 g were socially housed in temperature-and light-controlled facilities at UTMB with free access to food and water. Animals were acclimatized for one week before surgical procedures. No complications from surgery were observed and all animals survived until the scheduled experimental end point.

A Balb/C mice excision model was used with a cutaneous full-thickness injury made by a 6-mm diameter punch biopsy. The mice were 7-weeks old and 52 animals were used in the studies discussed herein.

The wounds were treated with an aqueous pharmaceutical composition prepared using a PV-10®-based rose bengal formulation. PV-10® is a sterile, non-pyrogenic 10% solution of disodium RB in 0.9% saline for injection. Using PV-10®, as its RB source, RB has been tolerated at a bolus dose of 1500 mg upon intravasation when delivered intratumorally; this equates to an exposure of approximately 300 mg/L in the bloodstream (300 μM RB).

Animals were administered the RB-containing PF-127-containing hydrogel every other day. A total of three animal treatment groups were studied along with two control groups. one group received vehicle plus light treatment, whereas the other group received vehicle without light. One treatment group of mice received 0.1% RB-containing hydrogel with irradiation for 30 minutes using monochromatic green light (MGL), λ=532 nm at a distance of 2.5 cm that provided 235 mW/cm² and a dose of 35J/cm². A second treatment group also received 0.1% RB-containing hydrogel, but without MGL irradiation. The third treatment group received 0.01% RB-containing hydrogel without MGL irradiation.

Primary outcomes assessed safety through analysis of full complete blood count (CBC), comprehensive blood chemistry, body and organ weight. Secondary outcomes analyzed wound closure, epidermal water loss, erythema, and comprehensive wound histology.

In the primary outcome and measures and observations, the treated animals and a control group received a full CBC and comprehensive blood chemistry panel, as well as observations of body weight food consumption, and organ appearance. Secondarily, wound closure, epidermal water loss erythema and wound histology were examined.

### Full-Thickness Excision and Treatment

Before surgery, animals received buprenorphine (0.1 mg kg⁻¹) for pre-procedural analgesia, and inhaled isoflurane (0.2-1.0%) was continuously administered throughout the procedure. The dorsum of the animal was shaved, a depilatory cream was applied to completely remove all dorsal hair, and the area was scrubbed with betadine antiseptic. The animal was laid in the right lateral decubitus position and using forceps, the dorsal skin was stretched, and a 6-mm punch biopsy was used to create bilateral full-thickness cutaneous wounds. Next, a 0.5 mm-thick silicone ring was sutured in place through the panniculus carnosus around each wound to ensure wounds healed primarily through re-epithelialization.

Animals were then randomized into treatment groups as follows: (1) Saline control in the dark, (2) Saline control with MGL irradiation, (3) Hydrogel vehicle in the dark, (4) Hydrogel vehicle with MGL irradiation, (5) RB in the dark, and (6) RB with MGL irradiation. Treatment was applied directly to the wound bed (800 uL per wound) and spread using a sterile surgical spatula to ensure even and uniform thickness over the wound.

Animals were then placed in a separate light-sealed and heated enclosure in a sternal position to expose dorsal wounds and remained under general isoflurane anesthesia. All other exposed portions of the body were then covered with sterile wrap to expose only the wounds. Animals were then irradiated with diffuse MGL (as described above), placed 20 cm directly above the dorsum at 90° from the midline, for 30 minutes. Those animals randomized to dark conditions underwent the same procedure, but MGL was not applied. After light treatment, 100% oxygen was delivered, and animals were allowed to recover until fully ambulatory. Animals were euthanized at 3-, 7-, and 14-days post-injury when wound healing parameters were assessed.

### Hydrogel and Rose Bengal Preparation

Preparations of all materials administered to animals were conducted in a sterile environment. Pluronic® F-127 (PF-127) is a synthetic hydrogel consisting of repeating propylene oxide and ethylene oxide polymers and was chosen as a vehicle for its thermoreversible gelation properties, RB solubility, and application as a topical gel formula on cutaneous wounds. [Chatterjee et al., “Dual-responsive (pH/temperature) Pluronic F-127 hydrogel drug delivery system for textile-based transdermal therapy”, Scientific Reports 9(1):11658 (2019)] PF-127 (P2443, MilliporeSigma, Burlington, MA) was diluted in pathogen-free microfiltered sterile water to a final concentration of 10% (wt/vol). More particularly, in 30 ml sterile water, 3.0 grams of F-127 are added and the mixture is shaken gently overnight (about 16 to 18 hours) at 4 degrees C. to form an aqueous gellant composition. Once the F-127 solution (gellant composition) is ready, the PV-10 solution is incorporated into the gellant composition to obtain the concentration needed for the study (either 0.1% or 0.01%). Gentle shaking at 4 degrees C. is continued overnight (about 16 to 18 hours). This solution is liquid at low temperatures <26 degrees and gelatinizes at body temperature when applied to the wound bed.

This formulation was liquid at ambient room temperatures (=26° C.) and formed a gel approaching mouse body temperature (=33° C.). The RB-containing aqueous pharmaceutical compositions so prepared and vehicle solutions were maintained at 4° C. and protected from light exposure until used in the reported studies. The aqueous pharmaceutical compositions were loaded into sterile 1 ml syringes and kept refrigerated until used for treatment. The cold hydrogel was applied to the wounds dropwise and flattened during the gelatinization process using a sterile spatula. Some of the gelled composition was removed by adherence to the dressing when the dressings were changed, with as much as possible of the remaining gelled composition in the wound being carefully removed with a similar sterile spatula Efforts were made to try not to perturb the wound bed or any granulation tissue in place. The wound treated again as above.

The sol-gel transition of drug-loaded formulations and PF-127 only are determined by formulations in 20 ml capped glass tubes and is checked at different temperatures in the range of 5-50° C. with increments of 1° C./stage inside thermostatic oven (ESCO Isotherm, Forced Convection Laboratory Oven). The temperature and flowability of the solutions inside the tubes are checked at each stage to determine the temperature at which the sample stops flowing inside the tube after tube inversion.

### Transepidermal Water Loss, Wound Erythema, and Closure

At each time point prior to euthanasia, transepidermal water loss (TEWL) and wound erythema indices were measured using the validated DermaLab combo system (CortexTechnologies, Hadsund, Denmark). Animals were placed under general anesthesia and a 10-mm diameter TEWL probe was placed directly over each wound ensuring uniform coverage and forming a seal immediately around the wound. Following environmental normalization to ambient temperature and relative humidity, TEWL was calculated at g m⁻¹ hr⁻¹. A minimum of three TEWL measurements were obtained for each full-thickness wound and uninjured skin area. A color sensitive 7-mm diameter probe, insensitive to ambient light, was then placed over each wound to measure erythema; four measures were obtained for each wound. All wound erythema calculations were normalized to uninjured skin and reported as an index value over time.

After initial surgery and at each study time point post-injury, color digital images were obtained for each wound. A digital camera was placed at a perpendicular angle (90°) and 15 cm from the center of each wound and uniform diffuse white light was used to obtain each image. For scale calculations, a metric ruler was placed at 15 cm from the camera lens and three images were obtained for each wound. Wound planimetry was calculated using ImageJ software (ver. 1.53) by tracing wound edges yielding a surface area in mm³. Wound closure was calculated as follows:

$$\% Original Wound Area = \text{} [ ( Original Wound Area - Current Wound area ) \text{} / Original Wound area ] \times 100$$

### Wound Healing Assessment

### Tissue Sampling

Following functional and planimetric measurements, animals were euthanized and the dorsal skin, inclusive down to underlying facia and dorsal musculature, was excised; care was employed to not disrupt any open wound post-mortem and maintain tissue integrity. The left-side wound and surrounding uninjured skin was directly transferred into a flat position histology cassette and the right-side wound was immediately snap-frozen in liquid nitrogen for reactive oxygen species and molecular analyses. Histology cassettes were then immersed in 10% neutral buffer formalin for 72 hours and subsequently placed in 70% ethanol thereafter. Uninjured skin tissue for each animal was also collected and preserved for future analyses.

### Tissue Histology

Skin/wound samples were processed through graded alcohols up to 100% ethanol and in subsequent changes of xylene to remove water. Tissues were then embedded in paraffin. Samples were then transversely cut to 4 um sections and mounted to slides, cleared in xylene, and then rehydrated in pure water in preparation for staining.

### H&E, Trichrome Staining, and Wound Healing Histological Assessment

Following rehydration, sections were stained with hematoxylin and eosin (H&E). H&E Images were digitized at 20X total magnification using an Olympus BX45 microscope equipped with CellSens software (Evident Corp., Waltham, MA]; the same light settings were maintained for every image acquired. A research-trained histopathologist, blinded to the study, marked the distance between the wound borders and the length of the re-epithelialization zone at 14 days post-injury.

In an additional study, samples were stained in Masson's trichrome (KTMTRPT, American MasterTech, Lodi, CA, USA) for assessment of collagen deposition and organization, as well as general wound healing parameters. As before, brightfield images were acquired at both low- and high-power magnification and observations of the wound were noted by a histopathologist utilizing modified measures of validated methods to histologically assess wound healing. [Gupta et al., “Assessment of the histological state of the healing wound”, Plast Aesthet Res (2):239-242 (2015)]. Briefly, H&E- and Trichrome-stained tissues were assessed at 7- and 14-days post-injury for formation of granulation tissue, inflammatory infiltrate, collagen fiber organization, vascularization, and presence and quality of adnexal structures.

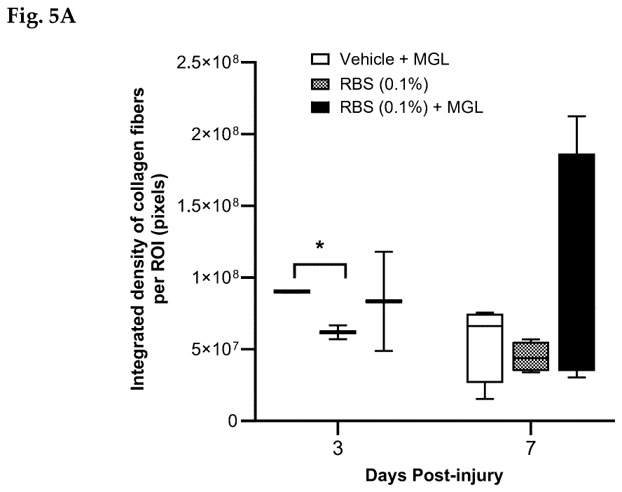

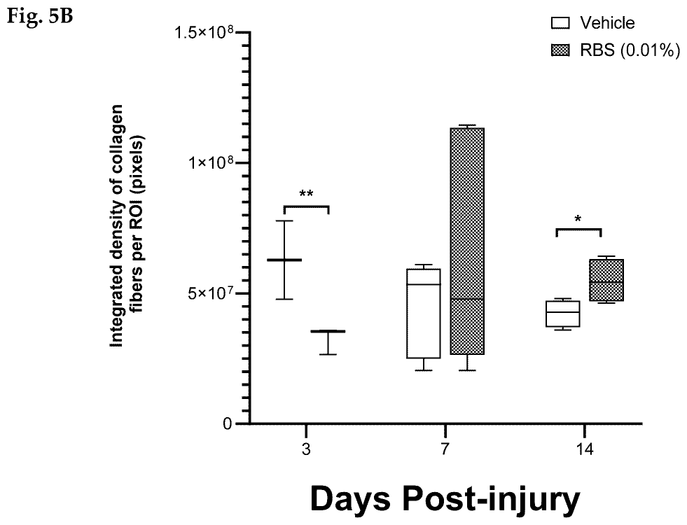

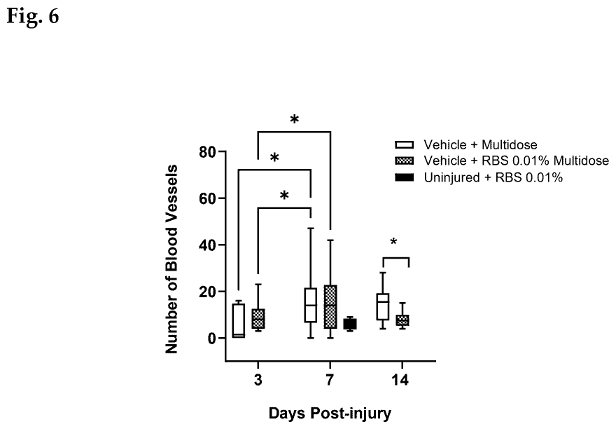

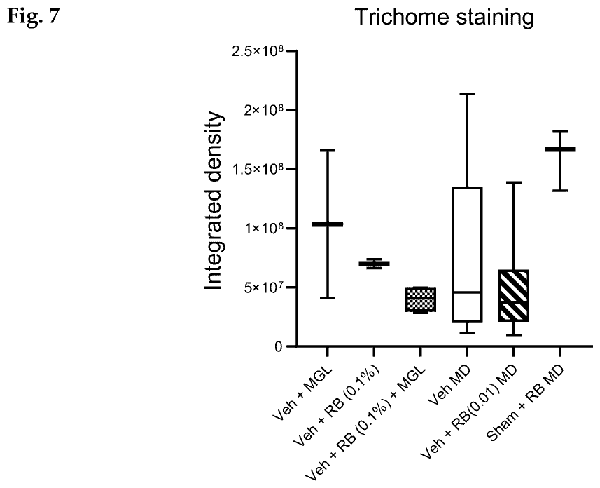

The method of collagen quantification was adapted from the teachings of Chen et al., Int J Clin Exp Med 10(10):14904-14910 (2017). This method is based on the open-resource image software ImageJ and an associated color deconvolution plugin. ImageJ is a free, downloadable, Java-based image processing program developed at the U. S. National Institutes of Health and the Laboratory for Optical and Computational Instrumentation (LOCI, University of Wisconsin). Original images of wounds were converted into red-blue-green (RGB) images, and these images were deconvolved by ImageJ using the color deconvolution plugin. The number of pixels stained by Trichrome dye providing evidence of collagen formation and healing. These area measurements are illustrated in FIGS. 5, 6 and 7.

### Wound Closure and ROS Generation by RB Phototherapy in Full-Thickness Injury

(a) Standardized digital images of full-thickness cutaneous injuries over 14 days in Balb/c mice track wound appearance, healing, and closure. Scale bar=2 mm. (b) Planimetric measurements of closure show photoactivated RB treatment significantly increased wound closure after 14 days post-injury compared with every other treatment group, p<0.05.

Additionally, both RB treatment with or without MGL irradiation significantly closes full-thickness wounds faster than saline-or vehicle-treated wounds at 3- and 7-days after initial injury. (c) RB phototherapy significantly increased tissue ROS at 3-days post-injury; this effect is sustained throughout 7-days compared to all other treatments and abates to nominal levels at day 14. Data are presented as mean±SD with n=3-6 animals per group

### Safety and Toxicity Methods

to determine the effects of rose bengal on blood parameters, blood samples were collected at euthanasia through cardiac puncture and were analyzed for complete blood count using an Abaxis VETSCAN® HM5 Hematology Analyzer Allied Analytic, LLC., Tampa, FL. The blood components analyzed included: white blood cells [WBC], lymphocytes [LYMPH], monocytes [MONO], neutrophils [NEUT], red blood cells [RBC], hemoglobin [HGB], hematocrit [HCT], mean corpuscle volume [MCV], mean corpuscle hemoglobin [MCH], mean corpuscle hemoglobin concentration [MCHC], platelets [PLT], and mean platelet volume [MPV]. Blood chemistry was also analyzed using an Abaxis VETSCAN® VS2 Chemistry Analyzer—Diagnostics to determine liver and Pancreas function through Albumin [ALB], Alkaline phosphatase [ALP], Alanine aminotransferase [ALT], Total bilirubin [tBIL], Total protein [TPRT], Globulin [GLOB], and Amylase [AMY]. Glucose & electrolyte levels were determined by measuring Glucose [GLU], Calcium [CA], Phosphorus [PHOS], Sodium [NA], and Potassium [K]. Kidney Function was assessed through Creatinine [CRE] and Blood urea nitrogen [BUN] levels. Body weight and gross examination of internal organs did not show any signs of toxicity.

### Statistical Analyses

All in vitro studies were repeated in triplicate, at minimum. Standard parametric tests, indicated in each experimental method, such as student's t-test, one-way ANOVA, or two-way ANOVA assessed differences between treatment groups over time in cases of normally distributed data. A post hoc Bonferroni multiple comparisons test assessed differences between treatment groups at each time point. Data normality was assessed through Shapiro-Wilk tests and Q-Q plots; results indicate that data were normally distributed for all data sets analyzed. Data analyses were performed in GraphPad Prism (ver. 9.4.0, La Jolla, California, USA). Significance was accepted at p<0.05.

1. A method of treating a skin wound that extends at least into the epidermal layer of the skin of a subject mammal that comprises
a) treating the wound in the substantial absence of actinic light by topical application to the wound of an aqueous pharmaceutical composition having dissolved or dispersed therein
i) a wound closure-assisting amount of rose bengal, a pharmaceutically acceptable salt thereof, an amide thereof whose nitrogen atom is unsubstituted, substituted with one or two C₁-C₄ alkyl groups that are the same or different or together with the amido nitrogen form a 5- or 6-membered ring, a C₁-C₄ alkyl ester thereof, an aromatic derivative thereof, wherein the aromatic derivative is an ester or amide formed from an alcohol or monosubstituted amine having a 5- or 6-membered aromatic ring, or a 5,6- or 6, 6-fused aromatic ring system that contains 0, 1 or 2 hetero ring atoms that are independently nitrogen, oxygen or sulfur, and
ii) a gel-inducing amount of a thickening agent that causes the aqueous pharmaceutical composition to gel at a temperature of about 33° to about 40° C.; and
b) covering the treated wound with a dressing that is opaque to actinic light in the area over the wound.

2. The method according to claim 1, wherein said aqueous pharmaceutical composition is a liquid at a temperature of about 15° to about 260.

3. The method according to claim 2, wherein said aqueous pharmaceutical composition contains about 15 to about 25 weight percent gellant.

4. The method according to claim 3, wherein said gellant amount includes a primary gellant and up to a total of about 20 weight percent of one or more secondary gellants.

5. The method according to claim 4, wherein said primary gellant is poloxamer 407.

6. The method according to claim 4, wherein said aqueous pharmaceutical composition contains up to about 5 weight percent of one or more secondary gellants.

7. The method according to claim 4, wherein said one or more secondary gellants is selected from the group consisting of chitosan, sodium alginate, gellan gum, κ-carrageenan, sodium carboxymethylcellulose, methyl cellulose, hydroxypropyl methylcellulose, hydroxyethyl cellulose, hydroxypropyl cellulose, polycarbophil, carbomer, and mixtures thereof.

8. The method according to claim 1, wherein said aqueous pharmaceutical composition is a liquid at a temperature of about 45° to boiling.

9. The method according to claim 1, wherein said treating step is repeated multiple times over a period of about 10 to about 15 days.

10. The method according to claim 1, wherein said wound extends at least in part through the epidermal layer of the skin into the dermal layer.

11. The method according to claim 1, wherein said wound extends into the subcutaneous tissue.

12. A method of treating a skin wound that extends at least into the dermal layer of the skin of a subject mammal that comprises
a) treating the wound in the substantial absence of actinic light by topical application to the wound of an aqueous pharmaceutical composition
i) having dissolved or dispersed therein present 0.05 to about 0.001% wt/vol rose bengal, a pharmaceutically acceptable salt thereof, an amide thereof whose nitrogen atom is unsubstituted, substituted with one or two C₁-C₄ alkyl groups that are the same or different or together with the amido nitrogen form a 5- or 6-membered ring, a C₁-C₄ alkyl ester thereof, an aromatic derivative thereof, wherein the aromatic derivative is an ester or amide formed from an alcohol or monosubstituted amine having a 5- or 6-membered aromatic ring, or a 5,6- or 6,6-fused aromatic ring system that contains 0, 1 or 2 hetero ring atoms that are independently nitrogen, oxygen or sulfur, wherein said wt/vol percentage is based on rose bengal disodium, and
ii) one or more gellants dissolved or dispersed therein that result in said pharmaceutical composition being a liquid at a temperature of about 15° to about 26° C., and a gel at a temperature of about 330 to about 40° C.;
b) covering the wound with a dressing that is opaque to actinic light in the area over the wound; and
c) wherein said treatment is repeated multiple times over a period of about 10 to about 15 days.

13. The method according to claim 12, wherein said treatment is repeated daily.

14. The method according to claim 13, wherein said treatment is repeated every other day.

15. The method according to claim 12, wherein said one or more gellants is present in said aqueous pharmaceutical composition at about 15 to about 25 weight percent of said composition.

17. The method according to claim 16, wherein said of one or more gellants are comprised of a primary gellant present at about 80 to about 100 weight percent of said one or more gellants, and one or more secondary gellants constituting the remainder the total gellant weight percentage present in said aqueous pharmaceutical composition.

18. The method according to claim 17, wherein poloxamer 407 is said primary gellant.

19. The method according to claim 18, wherein said one or more secondary gellants are present in said aqueous pharmaceutical composition.

20. The method according to claim 19, wherein said one or more secondary gellants are selected from the group consisting of chitosan, sodium alginate, gellan gum, κ-carrageenan, sodium carboxymethylcellulose, methyl cellulose, hydroxypropyl methylcellulose, hydroxyethyl cellulose, hydroxypropyl cellulose, polycarbophil, carbomer, and mixtures thereof.
